(19) United States
(12) Patent Application Publication
(10) Pub. No.: US 2026/0024663 A1
(43) Pub. Date: Jan. 22, 2026

(54) METHOD FOR PREDICTING OF MORTALITY RISK OR SEPSIS RISK AND DEVICE FOR PREDICTING OF MORTALITY RISK OR SEPSIS RISK USING THE SAME

(71) Applicant: AItrics Co., Ltd., Seoul (KR)

(72) Inventors: Sangchul Hahn, Seoul (KR); Kwangjoon Kim, Seoul (KR)

(73) Assignee: AItrics Co., Ltd., Seoul (KR)

(21) Appl. No.: 18/779,060

(22) Filed: Jul. 21, 2024

(51) Int. Cl. G16H 50/30

(52) U.S. Cl. CPC G16H 50/30

(57) ABSTRACT

Provided are a method for predicting a mortality risk or a sepsis risk, implemented by a processor, to predict an emergency situation, and a device using the same. The method for predicting a mortality risk or a sepsis risk includes: receiving biological signal data for a subject from a biological signal prediction device; generating a risk sequence for the subject based on the biological signal data, by using a risk sequence generation model configured to generate a risk sequence based on the biological signal data; and predicting a risk for the subject based on the risk sequence.

### BACKGROUND

### Field

The present disclosure relates to a method for predicting a mortality risk or a sepsis risk and a device for predicting a mortality risk or a sepsis risk using the same, and more particularly, to a method for predicting a mortality risk or a sepsis risk capable of predicting a risk based on biological signals of a subject and a device for predicting a mortality risk or a sepsis risk using the same.

### Description of the Related Art

Many patients who use medical services are easily exposed to fatal diseases and, in some cases, require continuous checkups of health conditions and appropriate actions corresponding thereto. Particularly for patients requiring special attention, such as serious patients in an intensive care unit of a hospital, it may be more important to continuously monitor patients' conditions.

Various prediction devices may be provided for serious patients to check up patients' conditions regarding matters related to disease progression or life support, such as pulses, blood pressures or respirations. These prediction devices measure the patients' conditions and display the results for medical staff to see. However, the conventional prediction devices have a limitation that the medical staff need to continuously monitor the prediction devices because the predicted information about the patients' conditions is provided on a display basis. That is, a risk prediction system based on the conventional prediction device has a problem in that it is not possible to take an appropriate precautionary action according to the predicted information in the case where continuous monitoring is not possible.

In particular, a patient who is in an intensive care unit with non-specific symptoms and various diseases tends to show a sharp change in condition. Thus, it may be more difficult to predict a prognosis using the conventional prediction device.

Accordingly, for a patient who has been provided with a medical service, particularly for a serious patient having a high degree of importance in prognosis prediction, there has continuously been a need to develop a new risk prediction system capable of predicting an emergency situation in advance and rapidly providing information associated with the emergency situation.

The description of the related art has been provided only to facilitate understanding of the present disclosure. The contents in the description of the related art should not be considered as the prior art.

### SUMMARY

The inventors of the present disclosure have noted that changes in biological signals would precede as a physiological response of a human body in advance before susceptibility to a certain disease is clinically suspected or an emergency situation such as mortality occurs.

In particular, in connection with a risk of a patient who is in a serious condition with a change in condition every hour, the inventors of the present disclosure have noted that changes in biological signal data that can be obtained in an intensive care unit may have correlation with potential serious conditions such as the onset of a particular disease, and even mortality.

As a result, the inventors of the present disclosure have recognized that the time-series biological signal data obtained for any period of time, such as temperatures, pulses, oxygen saturations, systolic blood pressures, diastolic blood pressures, mean blood pressures, and respirations, may be used to predict a risk in advance for a patient, particularly for a serious patient.

Furthermore, the inventors of the present disclosure have noted that clinical data on biological samples obtained from a patient may be closely correlated with a risk of disease onset, and further with a risk of mortality occurrence.

More specifically, the inventors of the present disclosure have recognized that biological test data, such as a Glasgow coma scale (GCS), an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level, may contribute to increasing an accuracy in risk prediction.

In particular, the inventors of the present disclosure have found that the biological test data may be closely correlated with the onset of sepsis, which causes mortality at a high rate for patients who are in a serious condition.

Meanwhile, the inventors of the present disclosure have noted not only the early prediction of the risk condition but also provision of an early alarm according to the result of prediction, as a way to overcome the limitations of the conventional risk prediction system.

Accordingly, the inventors of the present disclosure attempted to develop a new risk prediction system that is capable of predicting a risk using biological signal data and biological test data obtained from a patient and providing an alarm according to the result of prediction.

In particular, the inventors of the present disclosure attempted to develop a risk prediction algorithm for potential serious conditions such as the onset of a disease and mortality using biological signal data and further using biological test data.

The inventors of the present disclosure could expect that the development of such a risk prediction algorithm would make it possible to promptly detect a disease state of a patient, and further predict an emergency situation of the patient in advance, thereby making the treatment time earlier for the patient and as a result increasing a success in treatment. Furthermore, the inventors of the present disclosure have recognized that the development of risk prediction algorithms would increase a patient's survival rate, prevent complications, and reduce treatment costs.

Consequently, the inventors of the present disclosure have arrived at developing a new risk prediction system capable of predicting a mortality risk or a sepsis risk based on data obtained from a patient and providing an alarm according to the result of prediction.

More specifically, the risk prediction system of the present disclosure is configured to predict a risk situation at any time before the risk situation and provide the risk situation by using a risk sequence generation model configured to calculate a risk score based on biological signal data and further based on biological test data obtained from a subject such as a patient, and generate a risk sequence based thereon.

Furthermore, the risk prediction system of the present disclosure may be configured to provide an early alarm to medical staff or a guardian based on the result of prediction.

Meanwhile, the inventors of the present disclosure have noted the problem that when an alarm is provided simply based on a risk, for example, when an alarm is provided at a threshold risk value or above, false alarms may be frequently provided for a serious-condition patient.

Accordingly, the inventors of the present disclosure were able to apply to the risk prediction system an alarm sending model configured to determine whether to send an alarm in further consideration of biological signal data and biological test data for a subject.

An object to be achieved by the present disclosure is to provide a method for predicting a mortality risk or a sepsis risk including: generating a risk sequence based on biological signal data and further based on biological test data for a subject, and predicting a mortality risk or a sepsis risk based on the risk sequence.

Another object to be achieved by the present disclosure is to provide a method for predicting a mortality risk or a sepsis risk, using a risk alarming model configured to provide an alarm based on the risk for the subject.

Another object to be achieved by the present disclosure is to provide a device for predicting a mortality risk or a sepsis risk including: a receiving unit configured to receive biological signal data and further receive biological test data for a subject; and a processor connected to the receiving unit for communication and configured to predict a mortality risk or a sepsis risk for the subject based on various prediction models.

The objects of the present disclosure are not limited to the aforementioned objects, and other objects, which are not mentioned above, will be apparent to a person having ordinary skill in the art from the following description.

According to an aspect of the present disclosure, there is provided a method for predicting a mortality risk or a sepsis risk. The method for predicting a mortality risk or a sepsis risk, implemented by a processor, includes: receiving biological signal data for a subject; generating a risk sequence for the subject, by using a risk sequence generation model configured to generate a risk sequence based on the biological signal data; and predicting a mortality risk or a sepsis risk for the subject based on the risk sequence.

The objects of the present disclosure are not limited to the aforementioned objects, and other objects, which are not mentioned above, will be apparent to a person having ordinary skill in the art from the following description.

In order to solve the problems as described above, the present disclosure provides a method for predicting a mortality risk or a sepsis risk according to an exemplary embodiment of the present disclosure. A method for predicting a mortality risk or a sepsis risk, implemented by a processor, the method comprising: receiving biological signal data for a subject; generating a risk sequence for the subject, by using a risk sequence generation model configured to generate a risk sequence based on the biological signal data, and predicting a mortality risk or a sepsis risk for the subject based on the risk sequence.

According to a feature of the present disclosure, the receiving of biological signal data includes receiving at least one of the biological signal data for the subject selected from the group consisting of a temperature, a pulse, an oxygen saturation, a systolic blood pressure, a diastolic blood pressure, and a mean blood pressure. Furthermore, the predicting of a mortality risk or a sepsis risk includes predicting the mortality risk based on the risk sequence.

According to another feature of the invention, the receiving of biological signal data includes receiving the biological signal data a plurality of times in a predetermined unit of time, the generating of a risk sequence includes generating a risk sequence in the predetermined unit of time based on the biological signal data received the plurality of times in the predetermined unit of time, by using the risk sequence generation model. Furthermore, the predicting of a mortality risk or a sepsis risk includes predicting the mortality risk based on the risk sequence in the predetermined unit of time.

According to yet another feature of the present disclosure, the method for predicting a mortality risk or a sepsis risk, further comprising receiving at least one of biological test data for the subject selected from the group consisting of a Glasgow coma scale (GCS), an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level, the generating of a risk sequence further includes generating a sepsis risk sequence based on the biological signal data and the biological test data, by using the risk sequence generation model. Furthermore, the predicting of a mortality risk or a sepsis risk further includes predicting the sepsis risk based on the sepsis risk sequence.

According to yet another feature of the present disclosure, the receiving of biological test data includes receiving a maximum value, a minimum value, and an average value of the biological test data measured a plurality of times in a predetermined unit of time.

According to yet another feature of the present disclosure, the receiving of biological signal data includes receiving at least one of the biological signal data for the subject selected from the group consisting of a temperature, a pulse, an oxygen saturation, a systolic blood pressure, a diastolic blood pressure, and a mean blood pressure.

According to yet another feature of the present disclosure, the method for predicting a mortality risk or a sepsis risk, further comprising, after the predicting of a mortality risk or a sepsis risk, providing a risk alarm for the subject by using a risk alarming model.

According to yet another feature of the present disclosure, the method for predicting a mortality risk or a sepsis risk, further comprising, before the providing of a risk alarm, receiving biological test data for the subject, the risk sequence generation model is further configured to calculate a mortality risk score based on the biological signal data. Meanwhile, the risk alarming model includes: at least one of a first alarming model configured to output a vector value based on the mortality risk score calculated by the risk sequence generation model, a second alarming model configured to output a vector value based on the biological signal data, and a third alarming model configured to output a vector value based on the biological test data; and a fourth alarming model configured to determine whether to provide a mortality risk alarm based on the vector value outputted by the at least one of the first alarming model, the second alarming model, and the third alarming model.

According to yet another feature of the present disclosure, the method for predicting a mortality risk or a sepsis risk, further comprising, before the providing of a risk alarm, receiving drug administration recording or alarm transmission recording for the subject, the risk alarming model includes the first alarming model and the fourth alarming model. Furthermore, the first alarming model is further configured to output a vector value based on the mortality risk score, and the drug administration recording or the alarm transmission recording.

According to yet another feature of the present disclosure, the mortality risk is defined as a risk of mortality occurrence before a predetermined time, and the sepsis risk is defined as a risk of sepsis onset before the predetermined time.

According to yet another feature of the present disclosure, the risk sequence generation model is a model learning by: receiving learning biological signal data obtained for a specimen subject at a predetermined time before risk occurrence; generating a learning risk sequence based on the learning biological signal data; and predicting a risk for the specimen subject at any time before the risk occurrence based on the learning risk sequence.

According to yet another feature of the present disclosure, the specimen subject is a dead subject, and the predicting of a risk for the specimen subject includes predicting a mortality risk for the specimen subject based on the learning risk sequence.

According to yet another feature of the present disclosure, The method for predicting a mortality risk or a sepsis risk, further comprising receiving learning biological test data obtained for the specimen subject at a predetermined time before sepsis onset, the specimen subject is a subject suffering from sepsis, the generating of a learning risk sequence includes generating a learning sepsis risk sequence based on the learning biological test data and the learning biological signal data. Furthermore, the predicting of a risk for the specimen subject includes predicting a sepsis onset risk for the specimen subject based on the learning sepsis risk sequence.

According to yet another feature of the present disclosure, the receiving of biological signal data includes receiving a maximum value, a minimum value, and an average value of the biological signal data measured a plurality of times in a predetermined unit of time. Also, the generating of a risk sequence for the subject includes generating a risk sequence for the subject based on the maximum value, the minimum value, and the average value of the biological signal data, by using the risk sequence generation model.

According to yet another feature of the present disclosure, the method for predicting a mortality risk or a sepsis risk, further comprising receiving age data for the subject. At this time, the generating of a risk sequence for the subject further includes generating a risk sequence for the subject based on the biological signal data and the age data, by using the risk sequence generation model.

The present disclosure provides a system receiving biological signal data associated with a condition of a subject, and further with mortality of the subject, and predicting a risk based on the received data. Therefore, it is possible to promptly detect the onset of a disease in a subject, and it is also possible to provide information associated with life of the subject, thereby predicting an emergency situation.

In order to solve the problems as described above, the present disclosure provide a device for predicting a mortality risk or a sepsis risk according to another exemplary embodiment of the present disclosure, the device comprising: a receiving unit configured to receive biological signal data for a subject; and a processor connected to the receiving unit for communication. At this time, the processor is configured to generate a risk sequence for the subject by using a risk sequence generation model configured to generate a risk sequence based on the biological signal data, and predict a mortality risk or a sepsis risk for the subject based on the risk sequence.

According to a feature of the present disclosure, the receiving unit is configured to receive at least one of the biological signal data for the subject selected from the group consisting of a temperature, a pulse, an oxygen saturation, a systolic blood pressure, a diastolic blood pressure, and a mean blood pressure, and the processor is further configured to predict a mortality risk based on the risk sequence.

According to another feature of the present disclosure, the receiving unit is configured to receive the biological signal data a plurality of times in a predetermined unit of time, and the processor is further configured to generate a risk sequence in the predetermined unit of time based on the biological signal data received the plurality of times in the predetermined unit of time by using the risk sequence generation model, and predict a mortality risk based on the risk sequence in the predetermined unit of time.

According to yet another feature of the present disclosure, the receiving unit is further configured to receive at least one of biological test data for the subject selected from the group consisting of a glasgow coma scale, an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level. Also, the processor is further configured to generate a sepsis risk sequence based on the biological signal data and the biological test data by using the risk sequence generation model, and predict the sepsis risk based on the sepsis risk sequence.

According to yet another feature of the present disclosure, the processor is further configured to provide a risk alarm for the subject.

According to yet another feature of the present disclosure, the risk sequence generation model is further configured to calculate a mortality risk score based on the biological signal data, the receiving unit is further configured to receive biological test data for the subject. Furthermore, the risk alarming model includes: at least one of a first alarming model configured to output a vector value based on the mortality risk score calculated by the risk sequence generation model, a second alarming model configured to output a vector value based on the biological signal data, and a third alarming model configured to output a vector value based on the biological test data; and a fourth alarming model configured to determine whether to provide a mortality risk alarm based on the vector value outputted by the at least one of the first alarming model, the second alarming model, and the third alarming model.

According to yet another feature of the present disclosure, the receiving unit is further configured to receive a maximum value, a minimum value, and an average value of the biological signal data measured a plurality of times in a predetermined unit of time. At this time, the processor is further configured to generate a risk sequence for the subject based on the maximum value, the minimum value, and the average value of the biological signal data, by using the risk sequence generation model.

According to yet another feature of the present disclosure, the receiving unit is further configured to receive a maximum value, a minimum value, and an average value of the biological test data measured a plurality of times in a predetermined unit of time. At this time, the processor is further configured to generate a sepsis risk sequence for the subject based on the maximum value, the minimum value, and the average value of the biological test data, by using the risk sequence generation model.

According to yet another feature of the present disclosure, the receiving unit is further configured to receive age data for the subject. At this time, the processor is further configured to generate a risk sequence for the subject based on the biological signal data and the age data, by using the risk sequence generation model.

Accordingly, the present disclosure is capable of making it possible to make the treatment time earlier for the patient, thereby providing a good prognosis in treatment.

Further, the present disclosure is capable of further considering biological test data, which may be clinical data on biological samples obtained from the subject. Therefore, it is possible to predict, at a high precision, not only a mortality risk for the subject but also a risk of sepsis onset, which causes mortality at a high rate.

The present disclosure is capable of providing an early alarm according to the predicted mortality risk or sepsis risk. Therefore, it is possible to promptly take an action according to the predicted risk situation for a serious-condition subject such as a serious patient.

According to the present disclosure based on the early provision of an alarm, it is also possible to not only improve medical services, but also increase a survival rate, prevent complications, and reduce treatment costs.

According to the present disclosure, it is also possible to overcome the limitation of the risk prediction system that provides predicted information on a display basis and thus is not capable of providing an appropriate precautionary action according to the predicted information in the case where continuous monitoring is not possible.

In particular, the present disclosure uses an alarm sending model configured to determine whether to send an alarm in further consideration of biological signal data and biological test data for a subject. Therefore, it is possible to solve the problems following frequent alarm generation caused when the alarm is provided simply based on the risk.

The effects of the present disclosure are not limited to the aforementioned effects, and various other effects are included in the present specification.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1A exemplarily illustrates a risk prediction system using a device for predicting a mortality risk or a sepsis risk according to an exemplary embodiment of the present disclosure;

FIG. 1B illustrates a configuration of the device for predicting a mortality risk or a sepsis risk according to an exemplary embodiment of the present disclosure;

FIG. 2 exemplarily illustrates a procedure of a method for predicting a mortality risk or a sepsis risk according to an exemplary embodiment of the present disclosure;

FIGS. 3A to 3D illustrate learning data of a risk sequence generation model used in various embodiments of the present disclosure;

FIG. 3E exemplarily illustrates a procedure for preprocessing data inputted to the risk sequence generation model used in various embodiments of the present disclosure;

FIG. 3F exemplarily illustrates a configuration of the risk sequence generation model used in various embodiments of the present disclosure;

FIG. 3G illustrates learning data of a risk sequence generation model used in a device for predicting a mortality risk or a sepsis risk according to another exemplary embodiment of the present disclosure;

FIG. 3H exemplarily illustrates a configuration of the risk sequence generation model used in a device for predicting a mortality risk or a sepsis risk according to another exemplary embodiment of the present disclosure;

FIG. 4 exemplarily illustrates a configuration of a risk alarming model used in various embodiments of the present disclosure;

FIGS. 5A and 5B illustrate risk sequence generation results corresponding to an alive subject and a dead subject, respectively, based on a device for predicting a mortality risk or a sepsis risk according to an exemplary embodiment of the present disclosure;

FIG. 5C illustrates prediction results concerning mortality, based on the device for predicting a mortality risk or a sepsis risk according to an exemplary embodiment of the present disclosure;

FIG. 5D illustrates prediction results concerning the onset of sepsis, based on the device for predicting a mortality risk or a sepsis risk according to an exemplary embodiment of the present disclosure;

FIG. 6A illustrates prediction results concerning mortality, based on a device for predicting a mortality risk or a sepsis risk according to another exemplary embodiment of the present disclosure; and

FIG. 6B illustrates prediction results concerning the onset of sepsis, based on the device for predicting a mortality risk or a sepsis risk according to another exemplary embodiment of the present disclosure.

### DETAILED DESCRIPTION OF THE PREFERRED EMBODIMENT

The advantages of the present disclosure and the methods for accomplishment thereof will be apparent from exemplary embodiments described in detail below together with the accompanying drawings. However, the present disclosure is not limited to the following exemplary embodiments but may be implemented in variously different forms. The exemplary embodiments are provided for those skilled in the art to fully understand the present disclosure and the scope of the present disclosure. The present disclosure is defined only by the scope of the appended claims.

A shape, a size, a ratio, an angle, a number, etc. disclosed in the drawings for describing exemplary embodiments of the present disclosure are merely examples, and thus, the present disclosure is not limited to what are illustrated. In the following description, when the detailed description of the relevant known technologies is determined to unnecessarily obscure the gist of the present disclosure, the detailed description thereof will be omitted. The terms such as ‘comprise’, ‘have’, and ‘include’ used herein are generally intended to allow other components to be added unless the terms are used together with the term “only”. A component expressed in a singular form includes the component in a plural number unless explicitly stated otherwise.

Components are interpreted to include an error range even if not explicitly stated.

The features of various exemplary embodiments of the present disclosure may be partially or entirely coupled to or combined with each other. As fully understood by those skilled in the art, such features may be technically linked or operated together in various ways, and the exemplary embodiments may be implemented independently of or in association with each other.

For clarification in interpreting the present specification, the terms used herein will be defined below.

The term “subject” used herein may refer to an object for which an emergency situation occurrence, for example a risk of occurrence of mortality or a risk of the onset of a disease such as sepsis, is to be predicted. Meanwhile, the subject may be a ‘patient’ or a ‘serious patient’ in the specification but is not limited thereto. The subject may include any type of object for which a mortality risk or a sepsis risk is to be predicted.

The term “biological signal data” used herein may refer to data associated with a condition of a subject as a vital sign or life sign. The biological signal data may be associated with a mortality risk of a subject, and further with the onset of a disease such as sepsis. At this point, the biological signal data may be a temperature, a pulse, an oxygen saturation, a systolic blood pressure, a diastolic blood pressure, a mean blood pressure, and a respiration, which are measured by biological signal measurement equipment for the subject. Not being limited thereto, the biological signal data may include various measurement data associated with the health condition of the subject. Meanwhile, the biological signal data may be time-series data obtained from the subject at any time before the predicted mortality or sepsis.

The term “biological test data” used herein may be refer to clinical data on biological samples obtained from the subject. The biological test data may be associated with a mortality risk of a subject, and further with the onset of a disease such as sepsis. At this point, the biological test data may be a Glasgow coma scale (GCS), an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level, but are not limited thereto. Meanwhile, the biological test data may be a maximum value or a minimum value of the data obtained from the subject at any time before the predicted mortality or sepsis.

The term “risk sequence generation model” used herein may refer to a model configured to generate a risk sequence based on the biological signal data, and further based on the biological test data. In addition, the risk sequence generation model may be a model further learning to predict a mortality risk or a sepsis risk based on the risk sequence.

For example, the risk sequence generation model may be a model learning to calculate a risk score from a subject who has died or has sepsis based on the biological signal data and further based on the biological test data obtained at any time before mortality or sepsis occur, and generate a risk sequence based on the risk score. In addition, the risk sequence generation model may be a model further learning to predict a mortality risk or a sepsis risk based on the risk sequence.

Meanwhile, the risk sequence generation model of the present disclosure may be a prediction model based on a convolutional neural network (CNN)-based deep learning algorithm. For example, the risk sequence generation model may include an analysis module configured to analyze a risk through a Conv step, a BatchNorm and ReLu standardization step, and a MaxPool and Dropout step based on the inputted biological signal data, and an analysis module configured to analyze a risk through a Fully-connected step and BacthNorm step based on the biological test data. At this point, values outputted from the respective analysis modules in the risk sequence generation model may be combined through a Logit step for conversion into a probability logo so as to finally predict a mortality risk or a sepsis risk of a subject.

However, the structure of the risk sequence generation model of the present disclosure is not limited thereto and may be based on various algorithms that are capable of predicting a mortality risk or a sepsis risk on the basis of data about the subject. For example, the risk sequence generation model of the present disclosure may be a model based on a clustering algorithm, such as a k-Means or self-organizing map (SOM) algorithm, to form a pattern based on the biological signal data and/or the biological test data obtained from the subject.

The term “risk alarming model” used herein may a model configured to provide an alarm for a subject who is expected as a high-risk group, based on the risk sequence generated by the risk sequence generation model, the time-series data such as the biological signal data, and the biological test data, and further based on drug administration recording.

More specifically, the risk alarming model may be a model configured to learn expression forms of the risk score calculated in the risk sequence generation step and the time-series data including the biological signal data for the subject, and further including the biological test data, antibiotic/boosting agent administration recording, and alarm transmission recording, to determine whether to send an alarm. Accordingly, the risk alarming model of the present disclosure is capable of reducing false alarms when compared to provision of alarms simply based on the critical level of the risk sequence. That is, the risk alarming model is capable of providing an accurate alarm at the time when an action is required for a subject by further considering various data.

Meanwhile, the risk alarming model of the present disclosure may be a model based on a recurrent neural network (RNN) or CNN-based deep learning algorithm.

For example, the risk alarming model may include a plurality of models formed in multiple layers, including a plurality of alarming models each being composed of an independent RNN unit, and an alarming model composed of a layer determining whether to send an alarm based on a value outputted from each unit.

More specifically, the risk alarming model may include: a first alarming model of RNN unit configured to receive the mortality risk score calculated by the risk sequence generation model, time-series data including antibiotic/boosting agent administration recording, and alarm transmission recording for encoding into a multi-dimensional vector; a second alarming model of RNN unit configured to receive the time-series biological signal data for encoding into a multi-dimensional vector; and a third alarming model of RNN unit configured to receive the biological test data for encoding into a multi-dimensional vector. Furthermore, the risk alarming model may further include a fourth alarming model of Dense layer configured to output 0 or 1 based on the value outputted from each RNN unit to determine whether to send an alarm.

At this point, each of the first alarming model, the second alarming model, and the third alarming model may be configured as an RNN unit composed of a two-layer long short-term memory (LSTM). Further, data inputted to each of the first alarming model, the second alarming model, and the third alarming model may be converted into a linear matrix through an embedding layer before being inputted to each model. In addition, the value outputted through each model of RNN unit is inputted and concatenated finally to one dense layer, and the value is outputted by the dense layer as 0 or 1 through a sigmoid function to finally determine whether to send an alarm.

For example, the risk alarming model may be configured not to provide an alarm when the final value outputted by the fourth alarming model is 0, and configured to provide an alarm when the value outputted by the fourth alarming model is 1.

Meanwhile, according to various exemplary embodiments of the present disclosure, the risk alarming model of the present disclosure may include at least one of a first alarming model, a second alarming model, and a third alarming model configured for encoding as a vector value based on various time-series data, and a fourth alarming model configured to determine whether to provide a mortality risk alarm based on the vector value outputted by the at least one model.

That is, the risk alarming model may be configured to determine whether to send a risk alarm, based on not only the risk sequence but also the various data obtained from the subject. As a result, the device for predicting a mortality risk or a sepsis risk of the present disclosure is capable of sending an alarm more accurately by using the risk alarming model.

Preferably, the risk alarming model may include a first alarming model, a second alarming model, a third alarming model, and a fourth alarming model, but is not limited thereto.

Furthermore, the structure of the risk alarming model may be based on various machine learning algorithms, as long as an alarm is provided for a high-risk group based on the risk sequence and/or the data about the subject. For example, the risk alarming model of the present disclosure may also be configured to determine whether to send an alarm (for classification) based on the inputted risk sequence score, and further based on the biological signal data and/or the biological test data, on the basis of algorithms by machine learning of a randomized decision forest algorithm and a penalized logistic regression algorithm, and more various deep learning algorithms.

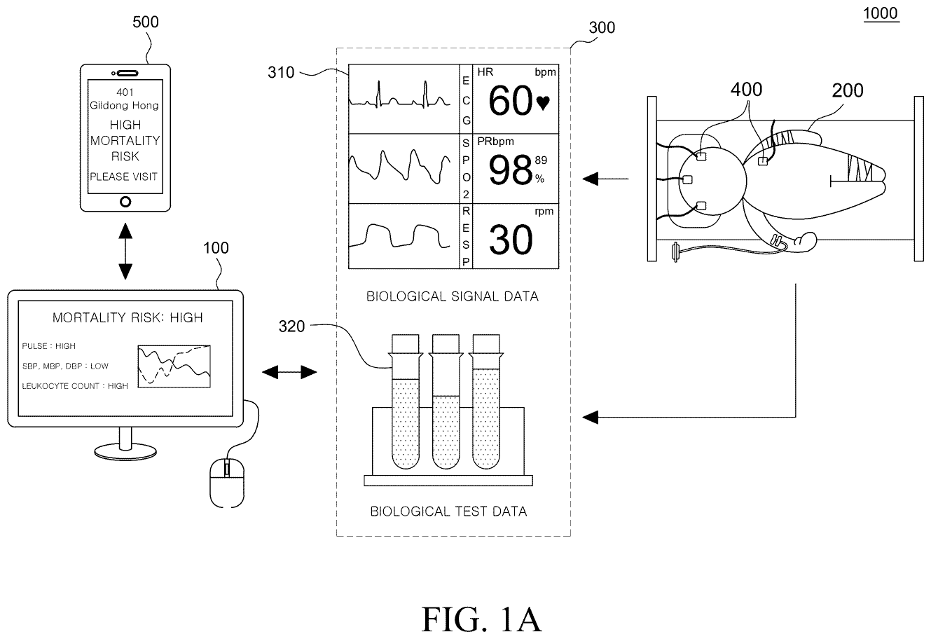

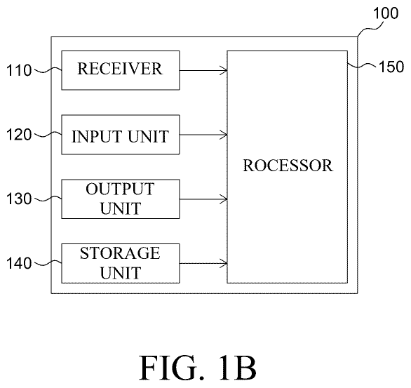

Hereinafter, a device for predicting a mortality risk or a sepsis risk according to an exemplary embodiment of the present disclosure and a risk prediction system using the same will be described in detail with reference to FIGS. 1A and 1B. FIG. 1A exemplarily illustrates a risk prediction system using a device for predicting a mortality risk or a sepsis risk according to an exemplary embodiment of the present disclosure. FIG. 1B illustrates a configuration of the device for predicting a mortality risk or a sepsis risk according to an exemplary embodiment of the present disclosure.

Referring to FIG. 1A, a risk prediction system 1000 according to an exemplary embodiment of the present disclosure includes a device 100 for predicting a mortality risk or a sepsis risk, data 300 obtained for a subject 200 including biological signal data 310 and biological test data 320, a biological signal measurement device 400 providing the biological signal data 310, and a medical staff device 500.

More specifically, in the risk prediction system 1000, the device 100 for predicting a mortality risk or a sepsis risk may be configured to receive the biological signal data 310 measured for the subject 200, generate a risk sequence for the subject 200, and predict a mortality risk based thereon. Furthermore, the device 100 for predicting a mortality risk or a sepsis risk may generate a risk sequence in further consideration of the biological test data 320 for the subject 200 and further predict a sepsis risk for the subject 200.

At this time, the biological signal measurement device 400 may be configured to transmit to the device 100 for predicting a mortality risk or a sepsis risk at least one biological signal data for the subject 200 selected from the group consisting of a temperature, a pulse, an oxygen saturation, a systolic blood pressure, a diastolic blood pressure, a mean blood pressure, and a respiration.

Meanwhile, in the risk prediction system 1000, the device 100 for predicting a mortality risk or a sepsis risk may further be configured to determine whether to send an alarm based on the risk sequence (or a risk score constituting the risk sequence) and further based on the biological signal data 310 and/or the biological test data 320 for a subject.

Accordingly, the device 100 for predicting a mortality risk or a sepsis risk may provide an alarm to the medical staff device 500 for a subject 200 who requires an action. Based thereon, the medical staff may recognize the alarm and provide a feedback, for example taking an action according to the symptoms of the subject 200.

More specifically, referring to FIG. 1B, the device 100 for predicting a mortality risk or a sepsis risk includes a receiving unit 110, an input unit 120, an output unit 130, a storage unit 140, and a processor 150.

Particularly, the receiving unit 110 may be configured to receive various data 300 including biological signal data 310 and biological test data 320 for the subject 200. For example, the receiving unit 110 may receive biological signal data 310 for the subject 200 including a temperature, a pulse, an oxygen saturation, a systolic blood pressure, a diastolic blood pressure, a mean blood pressure, and a respiration from the biological signal measurement device 400. Furthermore, the receiving unit 110 may further receive biological test data 320 for the subject 200 including a Glasgow coma scale (GCS), an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an APH level, and a hematocrit level.

According to a feature of the present disclosure, the receiving unit 110 may further be configured to transmit to the medical staff device 500 the prediction results for the subject 200 determined by the processor 150, which will be described later.

According to another feature of the present disclosure, the receiving unit 110 may further be configured to receive a maximum value, a minimum value, and an average value of the biological signal data measured a plurality of times in a predetermined unit of time. In addition, the receiving unit 110 may further be configured to receive a maximum value, a minimum value, and an average value of the biological test data measured a plurality of times in a predetermined unit of time.

According to another feature of the present disclosure, the receiving unit 110 may further be configured to receive age data for the subject.

The input unit 120 may be a keyboard, a mouse, a touch screen panel, or the like, but is not limited thereto. The input unit 120 may set the device 100 for predicting a mortality risk or a sepsis risk and instruct the device 100 for predicting a mortality risk or a sepsis risk to operate.

Meanwhile, the output unit 130 may display the biological signal data 310 or the biological test data 320 received by the receiving unit 110. The output unit 130 may also display a risk sequence generated by the processor 150 for the subject and visually display an emergency situation predicted by the processor 150. In addition, the output unit 130 may further be configured to output an alarm sound, if it is determined by the processor 150 to send an alarm.

The storage unit 140 may be configured to store the data 300 for the subject 200 received through the receiving unit 110, including the biological signal data 310 or the biological test data 320, and store an instruction of the device 100 for predicting a mortality risk or a sepsis risk set through the input unit 120. Furthermore, the storage unit 140 is configured to store a risk sequence for the subject 200 generated by the processor 150, which will be described later, and a predicted risk for the subject 200, and further store whether or not an alarm has been sent. However, the storage unit 140 is not limited to what has been described above and may store various information determined by the processor 150 for risk prediction.

The processor 150 may be a component for enabling the device 100 for predicting a mortality risk or a sepsis risk to provide accurate prediction results. For the accurate risk prediction, the processor 150 may be based on a biological signal risk sequence generation model learning to generate a risk sequence on the basis of the biological signal data 310 and/or the biological test data 320. For example, the processor 150 may generate a risk sequence based on the biological signal data 310 and/or the biological test data 320 using a risk sequence generation model, and predict a mortality risk or a sepsis risk based thereon.

In another exemplary embodiment, the processor 150 may further be configured to send an alarm according to the risk of the subject 200 using a risk alarming model. For example, the processor 150 may be based on a model configured to determine whether to send an alarm based on a risk score calculated by the risk sequence generation model, and further based on the biological signal data 310 and/or the biological test data 320. Accordingly, the processor 150 may provide an alarm by using the risk alarming model, when the subject 200 is predicted as a high-risk group. As a result, the medical staff may take a prompt action for the subject 200.

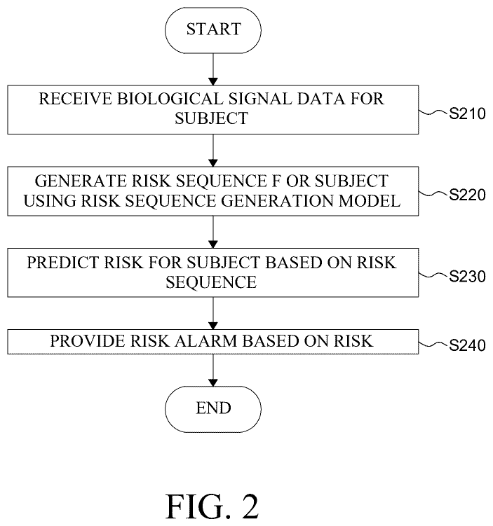

Hereinafter, a method for predicting a mortality risk or a sepsis risk according to an exemplary embodiment of the present disclosure will be described in detail with reference to FIG. 2. FIG. 2 illustrates a procedure of the method for predicting a mortality risk or a sepsis risk according to an exemplary embodiment of the present disclosure.

Referring to FIG. 2, the procedure for predicting a mortality risk or a sepsis risk according to an exemplary embodiment of the present disclosure is as follows: receiving biological signal data for a subject (S210); generating a risk sequence for the subject based on the received biological signal data, by using a risk sequence generation model (S220); predicting a risk for the subject based on the risk sequence (S230); and providing an alarm based on the risk (S240).

Specifically, in the receiving of the biological signal data (S210), biological signal data for a subject may be received. For example, in the receiving of the biological signal data (S210), at least one biological signal data for the subject selected from the group consisting of a temperature, a pulse, an oxygen saturation, a systolic blood pressure, a diastolic blood pressure, a mean blood pressure, and a respiration may be received.

Meanwhile, according to a feature of the present disclosure, in the receiving of the biological signal data (S210), biological signal data may be received a plurality of times in a predetermined unit of time. For example, in the receiving of the biological signal data (S210), biological signal data may be received from an admitted patient at an interval of one hour for 24 hours.

According to another feature of the present disclosure, in the receiving of the biological signal data (S210), a maximum value, a minimum value, and an average value of the biological signal data measured a plurality of times in a predetermined unit of time may be received.

According to another feature of the present disclosure, the method may further include receiving at least one of biological test data for the subject selected from the group consisting of a Glasgow coma scale (GSC), an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level.

According to another feature of the present disclosure, the method may further include receiving age data for the subject.

Next, in the generating of the risk sequence (S220), a risk sequence for the subject may be generated by using a risk sequence generation model learning to generate a risk sequence based on the biological signal data and/or the biological test data. At this point, the risk sequence generation model may be a model based on a deep learning algorithm forming a plurality of layers, configured to receive the biological signal data and/or the biological test data and calculate a risk score, and generate a risk sequence based thereon.

According to a feature of the present disclosure, in the generating of the risk sequence (S220), a mortality risk sequence may be generated by the risk sequence generation model based on the biological signal data.

According to another feature of the present disclosure, in the generating of the risk sequence (S220), a sepsis risk sequence may be generated by the risk sequence generation model based on the biological signal data and/or biological test data.

According to another feature of the present disclosure, in the generating of the risk sequence (S220), a risk sequence for the subject may be generated based on the maximum value, the minimum value, and the average value of the biological signal data, by using the risk sequence generation model.

According to another feature of the present disclosure, in the generating of the risk sequence (S220), a risk sequence for the subject may be generated based on the biological signal data and the age data, by using the risk sequence generation model.

Next, in the predicting of the risk (S230), a mortality risk may be probabilistically predicted based on the risk sequence. At this point, the risk prediction in the predicting of the risk (S230) may be performed by the above-described risk sequence generation model.

According to a feature of the present disclosure, in the predicting of the risk (S230), a mortality risk may be predicted based on the risk sequence. Furthermore, a sepsis risk may also be predicted based on the risk sequence. For example, in the predicting of the risk (S230), a mortality risk or a sepsis risk may be predicted at any time before a predetermined time.

Finally, in the providing of the risk alarm (S240), it may be determined whether or not an alarm is to be sent, based on the risk score obtained by the above-described risk sequence generation model, and further based on the biological signal data and/or the biological test data.

Meanwhile, in the providing of the risk alarm (S240), an alarm may be sent by a risk alarming model learning to determine whether to send an alarm based on the risk score, and further based on the biological signal data and/or the biological test data. For example, in the providing of the alarm (S240), the risk score, and further the biological signal data and/or the biological test data may be inputted to the risk alarming model to determine whether to send an alarm.

At this point, the risk alarming model may include a plurality of models formed in multiple layers, including a plurality of RNN units and a layer determining whether to send an alarm based on a value outputted from each unit.

For example, the risk alarming model may include: a first alarming model of RNN unit configured to receive time-series data including the mortality risk score calculated by the risk sequence generation model, antibiotic/boosting agent administration recording, and alarm transmission recording for encoding into a multi-dimensional vector; a second alarming model of RNN unit configured to receive the time-series biological signal data for encoding into a multi-dimensional vector; and a third alarming model of RNN unit configured to receive the biological test data for encoding into a multi-dimensional vector. Furthermore, the risk alarming model may further include a fourth alarming model of dense layer configured to output 0 or 1 based on the output value of each RNN unit to determine whether to send an alarm.

At this point, each of the first alarming model, the second alarming model, and the third alarming model may be configured as an RNN unit composed of a two-layer LSTM. Further, data inputted to each of the first alarming model, the second alarming model, and the third alarming model may be converted into a linear matrix through an embedding layer before being inputted to each model. In addition, the value outputted through each model of RNN unit is inputted and concatenated finally to the fourth alarming model of one dense layer, and the value is outputted by the fourth alarming model as 0 or 1 through a sigmoid function to finally determine whether to send an alarm.

As a result, according to the method of predicting a mortality risk or a sepsis risk according to an exemplary embodiment of the present disclosure, it is possible to predict an emergency situation, such as mortality or the onset of sepsis, for a subject, for example a serious patient, and provide an alarm at any time before the emergency situation occurs. Therefore, medical staff is able to take an effective action for the subject as the emergency situation varies.

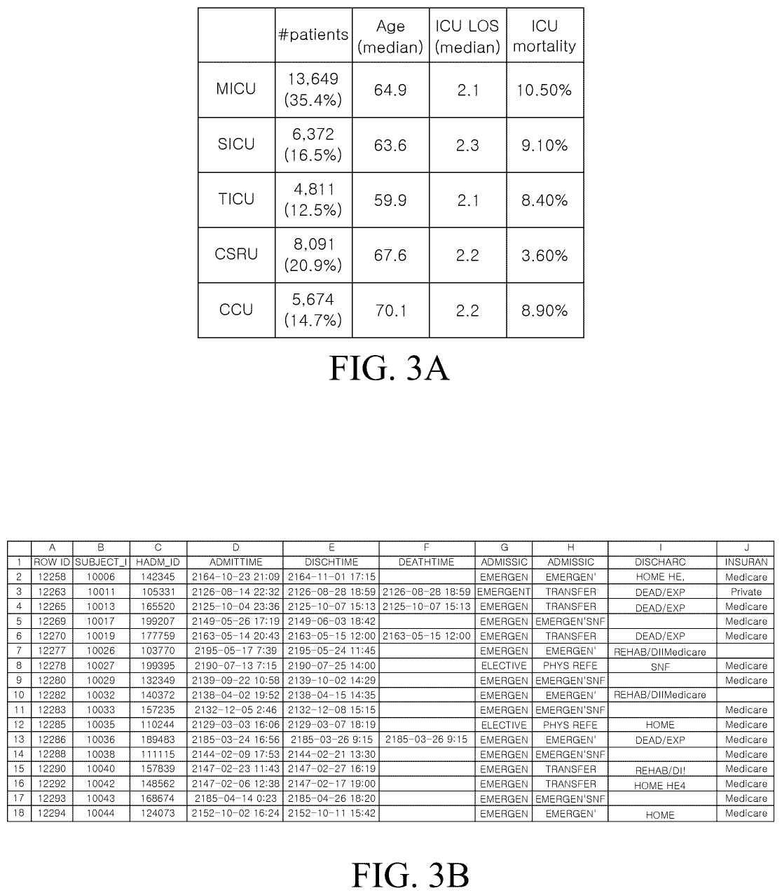

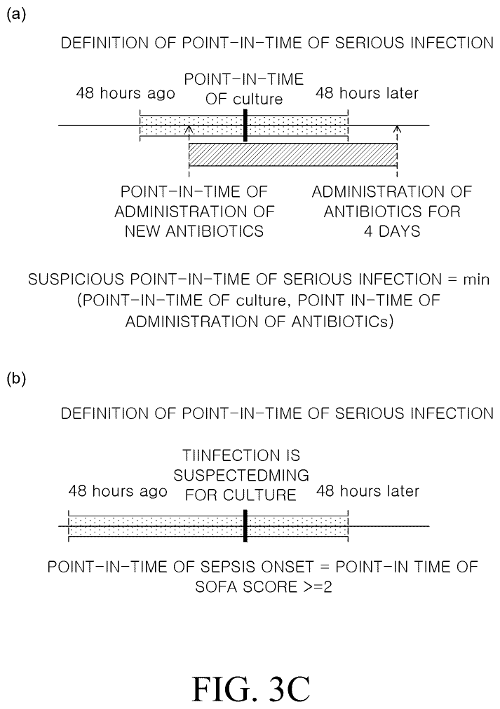

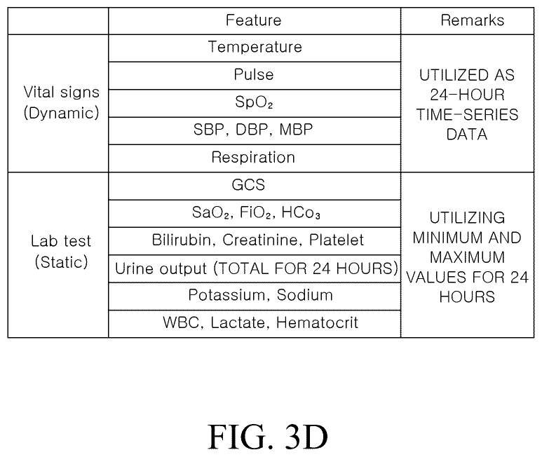

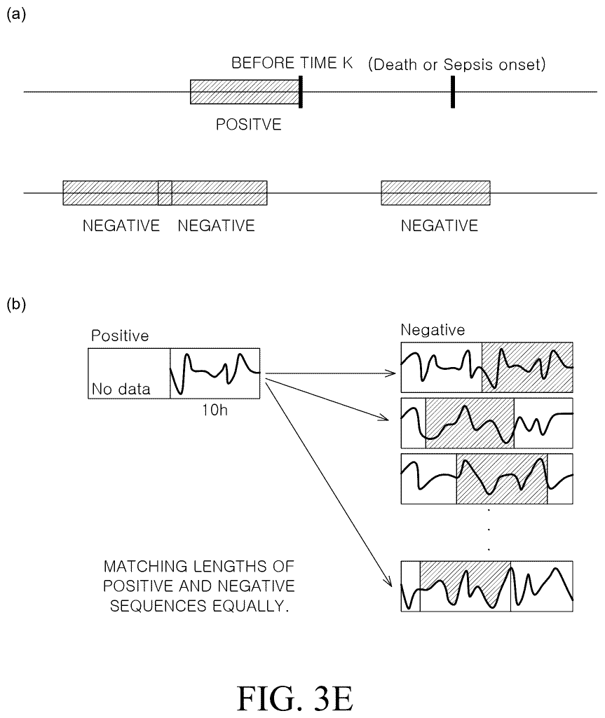

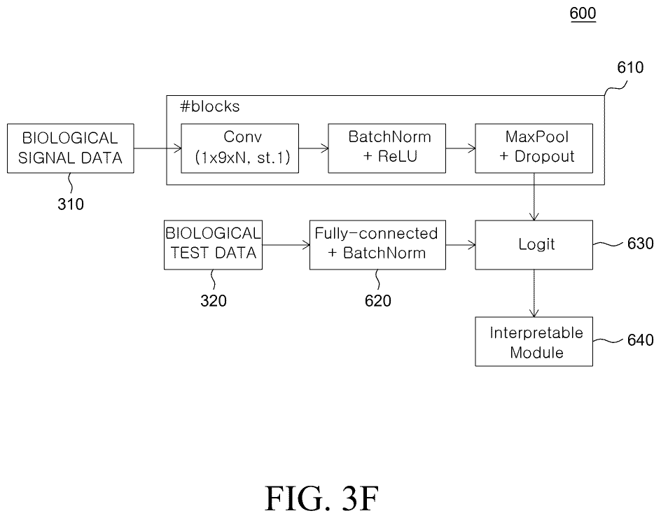

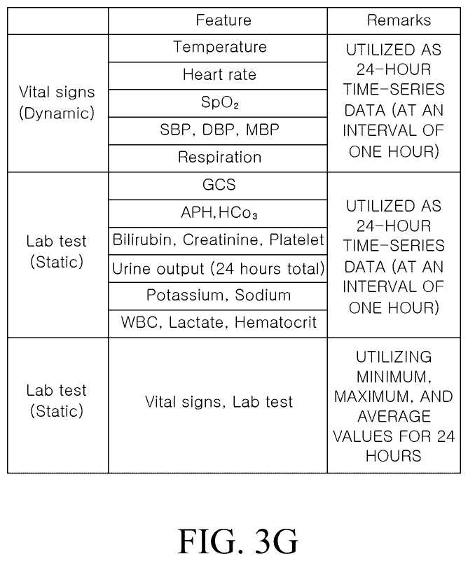

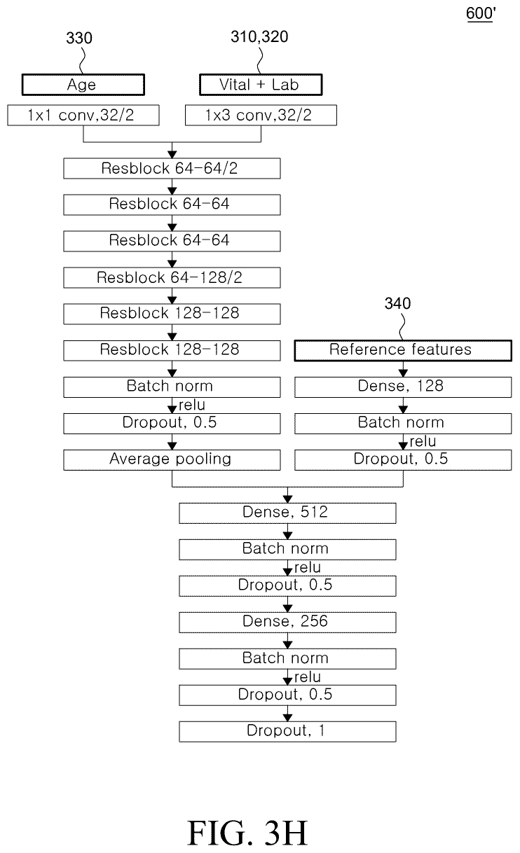

Hereinafter, a risk sequence generation model used in various exemplary embodiments of the present disclosure will be described in detail with reference to FIGS. 3A to 3F.

The risk sequence generation model used for the devices for predicting a mortality risk or a sepsis risk according to various exemplary embodiments of the present disclosure is a model learning to not only generate a risk sequence associated with a risk of mortality or the onset of sepsis, but also predict a risk based thereon. However, the risk sequence generation model is not limited thereto, and the generation of the risk sequence and the prediction of the risk may be performed in more various ways.

Furthermore, the risk sequence generation model of the present disclosure may receive more various data for a subject, and accordingly, predict more various diseases as well as the sepsis.

FIGS. 3A to 3D illustrate learning data of the risk sequence generation model used in various exemplary embodiments of the present disclosure.

Referring to FIG. 3A, as data for verifying the risk sequence generation model of the present disclosure, intensive care unit admission recording data obtained from the medical intensive care unit (MICU), the surgical intensive care unit (SICU), the trauma intensive care unit (TICU), the clinical safety research unit (CSRU), and the coronary care unit (CCU) for 38597 adult patients, which are included in Medical Information Mart for Intensive Care-III (MIMIC-III), have been used. For the learning data of the present disclosure, Severance intensive care unit (ICU) data have been used.

More specifically, referring to FIG. 3B, the intensive care unit admission recording data described above may include a patient identification (ID), an admission time, a discharge time, a mortality time, and the like. At this point, the risk sequence generation model of the present disclosure may learn to predict an overall risk related to mortality, with mortality during the admission as a label.

Referring to (a) of FIG. 3C, in a period of 48 hours before and after the point-in-time of incubation, or in a 4-day period from the point-in-time of antibiotic administration, the minimum points-in-time of the incubation and the antibiotic administration may be defined as suspicious points-in-time at which infection with a pathogen, which causes infection such as sepsis, is considered to have occurred. Referring to (b) of FIG. 3C, based on the sequential organ failure assessment (SOFA) score for assessing organ failure and prognosis for a period of 48 hours before and 24 hours after the suspicious point-in-time of infection, the point-in-time when the score is 2 or more may be defined as a point-in-time of sepsis onset. At this point, the risk sequence generation model of the present disclosure may learn to predict a sepsis risk, with the point-in-time of sepsis onset as a label.

Referring to FIG. 3D, learning data of the risk sequence generation model of the present disclosure is illustrated in detail. At this point, the learning data may include time-series data (dynamic data) of biological signals (vital signs) obtained for 24 hours, and biological test (lab test) data (static data) obtained by extracting minimum values and maximum values for 24 hours.

More specifically, the biological signal data may include temperatures, pulses, oxygen saturations (SpO₂), systolic blood pressures (SBP), diastolic blood pressures (DBP), mean blood pressures (MBP), and respirations obtained for a subject for 24 hours.

Further, the biological test data may include a minimum value and a maximum value for each of Glasgow coma scales (GCS), arterial oxygen saturations (SaO₂), fractions of inspired oxygen (FiO₂) concentration, bicarbonate ion (HCO₃) concentrations, bilirubin levels, creatinine levels, platelet counts, total urine outputs, potassium concentrations, sodium concentrations, white blood cell (WBC) counts, lactate concentrations, and hematocrit levels measured for 24 hours.

The risk sequence generation model of the present disclosure may learn to calculate a risk score based on the biological signal data and/or the biological test data described above, and generate a risk sequence based thereon. Furthermore, the risk sequence generation model may further learn to predict a mortality risk or a sepsis risk based on the risk sequence.

Meanwhile, the biological signal data and biological test data for learning the risk sequence generation model are not limited thereto, and the risk sequence generation model may learn to generate a risk sequence based on more various data and predict a mortality risk or a sepsis risk based thereon.

FIG. 3E exemplarily illustrates a procedure for preprocessing data inputted to the risk sequence generation model used in various embodiments of the present disclosure.

Referring to (a) of FIG. 3E, the learning data may include positive data and negative data. More specifically, the positive data may be data for any period of time before a predetermined time (time K) from the point-in-time of mortality or sepsis onset. In the meantime, the negative data may be obtained from data sampled for 24 hours from any point-in-time.

Referring to (b) of FIG. 3E, the positive data may have a certain length according to a preset period of time. At this point, the negative data may be adjusted to have the same length as the positive data, with respect to the data sampled for 24 hours from any point-in-time through the preprocessing procedure. For example, if the positive data is data obtained for 10 hours, the negative data may be corrected to have a length corresponding to 10 hours with respect to the data sampled for 24 hours in the preprocessing step.

This preprocessing procedure is not limited to the step of learning by the risk sequence generation model. For example, time-series biological signal data obtained from a subject for any period of time may be corrected through the preprocessing procedure such that each of the biological signal data has the same length as the others.

FIG. 3F exemplarily illustrates a configuration of the risk sequence generation model used in various embodiments of the present disclosure.

Referring to FIG. 3F, the risk sequence generation model 600 of the present disclosure may be a prediction model based on a convolutional neural network (CNN)-based deep learning algorithm. More specifically, the risk sequence generation model 600 may include an analysis module 610 configured to analyze a risk through a Conv step, a BatchNorm and ReLu standardization step, and a MaxPool step and Dropout step based on the inputted biological signal data 310, and an analysis module 620 configured to analyze a risk through a Fully-connected and BacthNorm step based on the biological test data 320. At this point, the value outputted from each analysis module 610 or 620 in the risk sequence generation model 600 is converted into a probability logo through a Logit module 630. Based thereon, a mortality risk or a sepsis risk for a subject is finally predicted by an interpretable module 640.

However, the structure of the risk sequence generation model 600 of the present disclosure is not limited thereto and may be based on various algorithms that are capable of predicting a mortality risk or a sepsis risk on the basis of the data about the subject. For example, the risk sequence generation model (600) of the present disclosure may be a model based on a clustering algorithm, such as a k-Means or SOM algorithm, to form a pattern on the basis of the biological signal data and/or the biological test data obtained from the subject.

Meanwhile, according to another exemplary embodiment of the present disclosure, the risk sequence generation model may learn based on more various learning data and may have more various structures.

Hereinafter, the risk sequence generation model used in a device according to another exemplary embodiment of the present disclosure will be described in detail with reference to FIGS. 3G and 3H.

Referring to FIG. 3G, learning data of the risk sequence generation model used in a device according to another exemplary embodiment of the present disclosure is specifically illustrated. At this point, the learning data may include time-series data (dynamic data) of biological signals (vital signs) obtained at an interval of one hour for 24 hours, time-series data (dynamic data) of biological tests (lab test) obtained at an interval of one hour for 24 hours, and reference feature data including a minimum value, a maximum value, and an average value for each of the biological signal data and the biological test data obtained at an interval of one hour for 24 hours.

More specifically, the biological signal data may include temperatures, heart rates, oxygen saturations (SpO₂), systolic blood pressures (SBP), diastolic blood pressures (DBP), mean blood pressures (MBP), and respirations that are obtained for a subject at an interval of one hour for 24 hours.

Further, the biological test data may include Glasgow coma scales (GCS), anterior pituitary hormone (APH) levels, bicarbonate ion (HCO₃) concentrations, bilirubin levels, creatinine levels, platelet counts, potassium concentrations, sodium concentrations, white blood cell (WBC) counts, lactate concentrations, and hematocrit levels that are measured at an interval of one hour for 24 hours.

As described above, the reference feature data may include a minimum value, a maximum value, and an average value for each of the biological signal data and the biological test data obtained at an interval of one hour for 24 hours.

According to another exemplary embodiment of the present disclosure, the risk sequence generation model of the present disclosure may further use an age of the subject as learning data to predict a sepsis risk or a mortality risk.

The risk sequence generation model of the present disclosure may learn to calculate a risk score based on the learning biological signal data and/or biological test data and/or reference feature data, which are described above, and generate a risk sequence based thereon. Furthermore, the risk sequence generation model may further learn to predict a mortality risk or a sepsis risk based on the risk sequence.

FIG. 3H exemplarily illustrates a configuration of the risk sequence generation model used in a device for predicting a mortality risk or a sepsis risk according to another exemplary embodiment of the present disclosure.

At this point, according to another exemplary embodiment of the present disclosure, the risk sequence generation model 600′ may include a plurality of layers to which the age data 330 is inputted together with the biological signal data 310 and the biological test data 320, and a plurality of layers to which the reference feature data 340 is inputted.

More specifically, the biological signal data 310 and the biological test data 320 inputted to a 1×3 convolution layer and the age data 330 inputted to a 1×1 convolution layer pass through six (6) residual (Res) block layers, a batch normalization (Batch norm) layer, a dropout layer, and an average pooling layer. At the same time, the reference feature data 330 inputted to a dense layer passes through a Batch norm layer and a dropout layer. Thereafter, all the data 310, 320 and 330 are inputted integrally to a dense layer and then pass through a Batch norm layer, a dropout layer, a dense layer, another Batch norm layer, another dropout layer, and another dense layer, thereby finally outputting a result value associated with a mortality risk or a sepsis risk. The risk sequence generation model 600′ in the structure described above may provide interpretability.

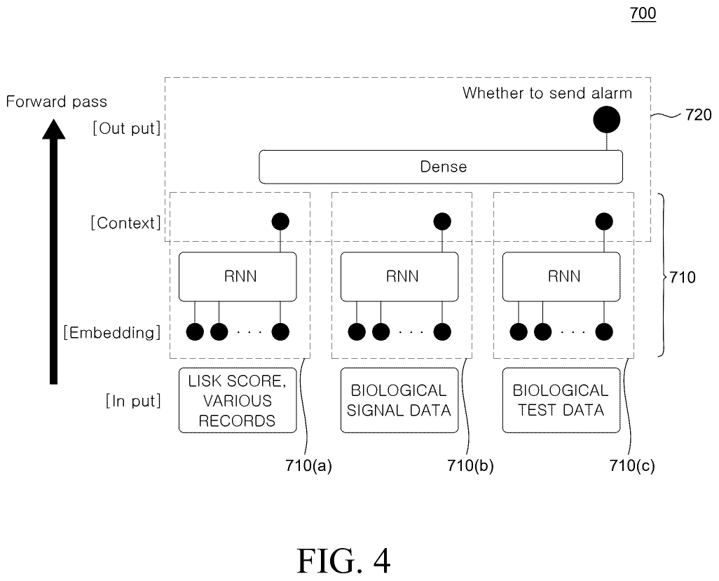

Hereinafter, a risk alarming model of the present disclosure will be described in detail with reference to FIG. 4. FIG. 4 exemplarily illustrates a configuration of a risk alarming model used in various embodiments of the present disclosure.

The risk alarming model used in various embodiments of the present disclosure may be a model based on a recurrent neural network (RNN) or CNN-based deep learning algorithm.

For example, the risk alarming model may be configured in a multilayer structure including a plurality of RNN units and a layer determining whether to send an alarm based on a value outputted from each unit. More specifically, the risk alarming model may include: a long short-term memory (LSTM) unit configured to receive the mortality risk score calculated by the risk sequence generation model, and provide an output value associated with whether to provide an alarm; an RNN unit configured to receive the biological signal data, and provide an output value associated with whether to provide an alarm; and an RNN unit configured to receive the biological test data, and provide an output value associated with whether to provide an alarm. Furthermore, the risk alarming model may further include a dense layer (Fully connected layer) configured to output 0 or 1 based on the value outputted from each LSTM to determine whether to send an alarm.

Meanwhile, the device for predicting a mortality risk or a sepsis risk according to various exemplary embodiments of the present disclosure may send an alarm by using a risk alarming model configured to provide an alarm for a subject expected as a high-risk group based on the risk sequence generated by the risk sequence generation model.

Referring to FIG. 4, the risk alarming model 700 of the present disclosure may be a model based on an RNN or CNN-based deep learning algorithm.

More specifically, the risk alarming model 700 may be a model with two layers formed by a first alarming model 710(a), a second alarming model 710(b) and a third alarming model 710(c), and a fourth alarming model 720 of dense layer determining whether to send an alarm based on the value outputted from each of the plurality of alarming models 710.

The risk alarming model 700 may include: a first alarming model 710(a) of RNN unit configured to receive time-series data including the risk score calculated by the risk sequence generation model described above, administration recording of drug such as antibiotic/boosting agents, and transmission recording of previously-transmitted alarms (binary vector) for encoding into a multi-dimensional vector; a second alarming model 710(b) of RNN unit configured to receive the time-series biological signal data for encoding into a multi-dimensional vector; and a third alarming model 710(c) of RNN unit configured to receive the time-series biological test data for encoding into a multi-dimensional vector. Furthermore, the risk alarming model 700 may further include a fourth alarming model 720 of dense layer configured to combine the respective values lastly outputted from the plurality of alarming models 710 and output 0 or 1 through a sigmoid function so as to finally determine whether to send an alarm.

At this point, each of the first alarming model 710(a), the second alarming model 710(b), and the third alarming model 710(c) may be configured as an RNN unit composed of a two-layer long short-term memory (LSTM). Further, data inputted to each of the first alarming model 710(a), the second alarming model 710(b), and the third alarming model 710(c) may be converted into a linear matrix through an embedding layer before being inputted to each model.

More specifically, four (4) four-dimensional time-series data up to a specific point in time (X₁∈ [symbol] inputted to an input layer, including risk scores, antibiotic administration recording, boosting agent administration recording, and alarm transmission recording, are converted into a linear matrix through an embedding layer (x_{e,1}=W_{e,1}_{^{x}}₁), and then inputted to the first alarming model 710(a). Furthermore, (seven (7) biological signal data up to a specific point in time (x₂∈ [symbol] ) inputted to an input layer may be converted into a linear matrix through an embedding layer (x_{e,2}=W_{e,2}_{^{x}}₂), and then inputted to the second alarming model 710(b). In addition, three (3) biological test data up to a specific point in time (x₃∈ [symbol] ) inputted to an input layer may be converted into a linear matrix through an embedding layer (x_{e,3}=W_{e,3}_{^{x}}₃, and then inputted to the third alarming model 710(c).

Here, x₁, x₂, and x₃ each denote original data (the four-dimensional time-series data, the biological signal data, and the biological test data), x_{e,1}, x_{e,2} and x_{e,3} each denote data converted into a linear matrix. R denotes an Euclidean space. t represents a sequence length of each original data, and W denotes a linear matrix defining an embedding layer.

At this piont, the seven (7) biological signal data inputted to the second alarming model 710(b) may be a systolic blood pressure (SBP), a diastolic blood pressure (DBP), a mean blood pressure (MBP), a pulse, a respiration, a heart rate (HR), and an oxygen saturation (SpO₂), and the three (3) biological test data inputted to the third alarming model 710(c) may be a bilirubin level, a lactate concentration, and a creatinine level. However, the seven (7) biological signal data and the three (3) biological test data are not limited thereto.

Next, the four-dimensional time-series data inputted to the first alarming model 710(a) may be encoded into a multi-dimensional vector through a context layer based on the following Equation 1.

$${h}_{{t}_{1}}^{1} = RNN ( {x}_{1} ) , {h}_{{t}_{1}}^{1} ∈ {ℝ}^{{d}_{1}} \qquad [ Equation 1 ]$$

The time-series data of biological signals inputted to the second alarming model 710(b) may be encoded into a multi-dimensional vector through a context layer based on the following Equation 2.

$${h}_{{t}_{2}}^{2} = RNN ( {x}_{2} ) , {h}_{{t}_{2}}^{1} ∈ {ℝ}^{{d}_{2}} \qquad [ Equation 2 ]$$

In addition, the time-series data of biological test data inputted to the third alarming model 710(c) may be encoded into a multi-dimensional vector through a context layer based on the following Equation 3.

$${h}_{{t}_{3}}^{3} = RNN ( {x}_{3} ) , {h}_{{t}_{3}}^{1} ∈ {ℝ}^{{d}_{3}} \qquad [ Equation 3 ]$$

Here, h denotes a hidden variable generated through a RNN at the last point in time, and d represents a dimension of a corresponding latent variable.

Lastly, when a vector value outputted finally from each of the plurality of alarming models 710 is inputted to the fourth alarming model 720 of dense layer, 0 or 1 is outputted through a sigmoid function based on the following Equation 4.

$$y = sigmoid ( {w}_{0}^{⊤} [ {h}_{t 1}^{1} ; {h}_{t 2}^{2} ; {h}_{t 3}^{3} ] ) , {w}_{o} ∈ {ℝ}^{{d}_{1} + {d}_{2} + {d}_{3}} \qquad [ Equation 4 ]$$

Here, Wₒ denotes a linear vector for determining whether to send an alarm. The risk alarming model may be configured not to provide an alarm if the value finally outputted by the fourth alarming model is 0, and configured to provide an alarm if the value outputted by the fourth alarming model is 1.

According to another feature of the present disclosure, the risk alarming model 700 may be a model configured to calculate a loss function on the basis of cross entropy loss and weight decay of L2 regularization, based on an objective function of the following Equation 5, and learn expression forms of the time-series data based thereon.

$$𝒥 ( θ ) = \frac{1}{N} {\sum }_{i = 1}^{N} [ {y}_{i} log {\hat{y}}_{i} + ( 1 - {y}_{i} ) log ( 1 - {\hat{y}}_{i} ) ] + {λ}_{1} {‖ W ‖}_{2}^{2} , \\ {\hat{y}}_{i} = f ( {x}_{1} , {x}_{2} , {x}_{3} ; W ) \qquad [ Equation 5 ]$$

Here, ŷᵢ denotes whether the model predicts an alarm to be sent (binary variable, 0 or 1).

The risk alarming model 700 having such a configuration is capable of reducing false alarms when compared to provision of alarms simply based on the critical level of the risk sequence. That is, the risk alarming model 700 is capable of providing an alarm at the time when an action is required for a subject, by further considering various data.

Meanwhile, the structure of the risk alarming model 700 of the present disclosure is not limited thereto, and may be based on various machine learning algorithms as long as an alarm is provided for a high-risk group based on the risk sequence and/or the data about the subject. For example, the risk alarming model of the present disclosure may also be configured to determine whether to send an alarm (for classification) based on the inputted risk sequence score, and further based on the biological signal data and/or the biological test data, on the basis of algorithms by machine learning of a randomized decision forest algorithm and a penalized logistic regression algorithm, and more various deep learning algorithms.

Hereinafter, a method of predicting a mortality risk or a sepsis risk according to an exemplary embodiment of the present disclosure and a result of evaluating a device using the same will be described with reference to Example 1 below.

### EXAMPLE 1

### First Evaluation of Risk Sequence Generation Model Applied to Various Exemplary Embodiments of Present Disclosure

The evaluation was conducted on a risk sequence generation model, which was configured to assess a sepsis risk or a mortality risk based on biological signal data and biological test data.

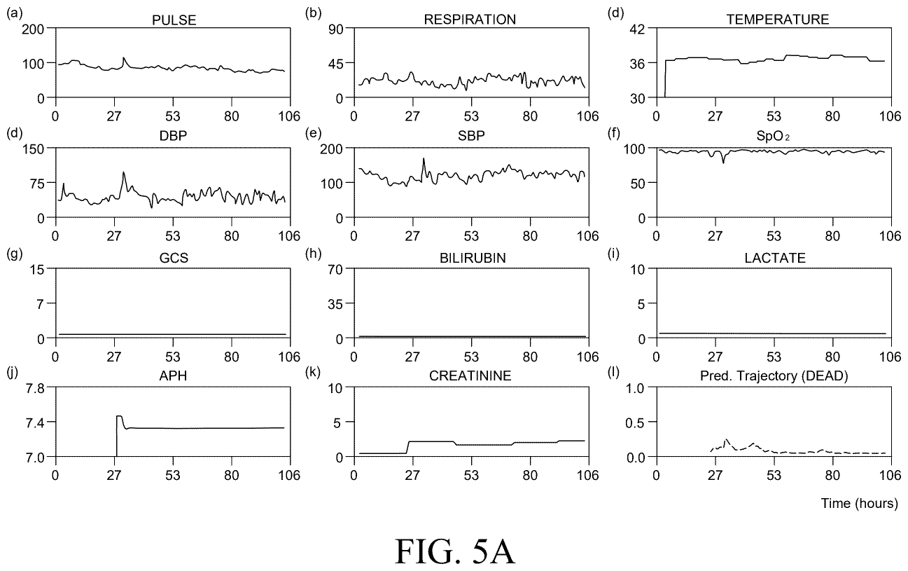

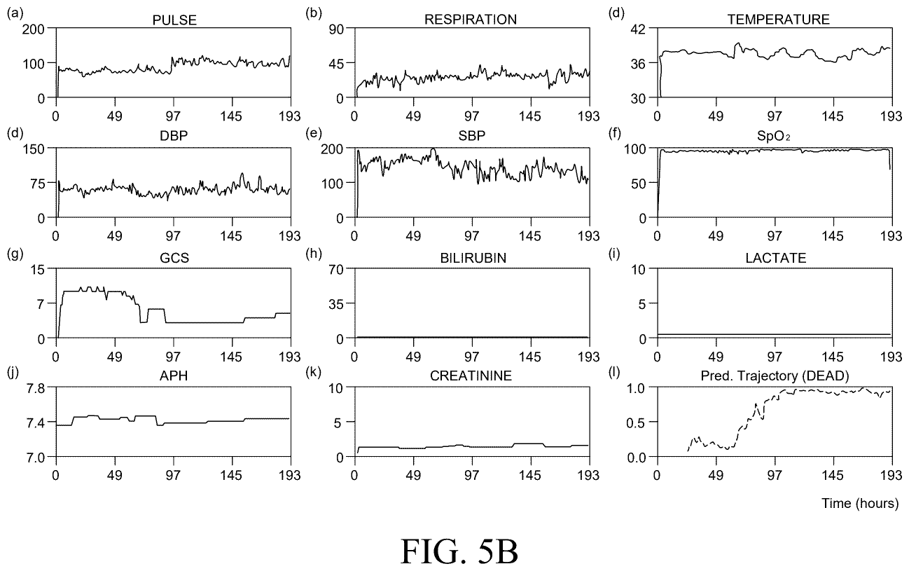

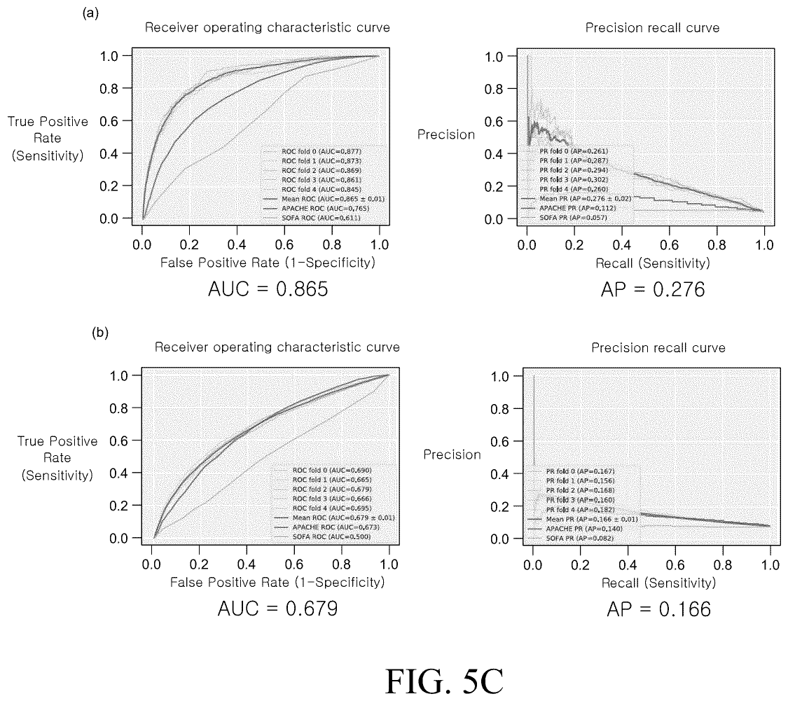

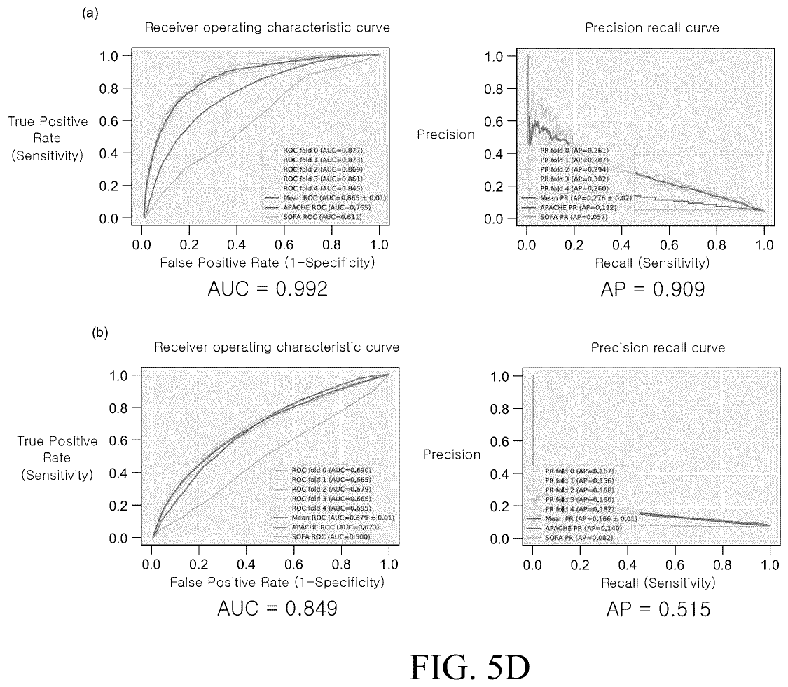

FIGS. 5A and 5B illustrate risk sequence generation results corresponding to an alive subject and a dead subject, respectively, based on a device for predicting a mortality risk or a sepsis risk according to an exemplary embodiment of the present disclosure.

Referring to (a), (b), (c), (d), (e), (f), (g), (h), (i), (j) and (k) of FIG. 5A, sequences of biological signal data and biological test data generated for an alive subject are shown. Referring to (l) of FIG. 5A, a risk sequence generated by the risk sequence generation model used in various exemplary embodiments of the present disclosure is shown. More specifically, it is shown in (l) of FIG. 5A that when the subject is alive, the risk score calculated based on the biological signal data and the biological test data is close to 0. That is, a subject having a low mortality risk may be predicted by the risk sequence generation model to have a low risk.

In contrast, referring to (a), (b), (c), (d), (e), (f), (g), (h), (i), (j) and (k) of FIG. 5B showing sequences of biological signal data and biological test data generated for a dead subject, the data values are greatly changed when compared to those for the alive subject. Referring to (l) of FIG. 5B, it is shown that the risk score in the risk sequence generated by the risk sequence generation model for the dead subject is close to 1. That is, a subject having a high mortality risk may be predicted by the risk sequence generation model to have a high-risk.

In various exemplary embodiments of the present disclosure, the device for predicting a mortality risk or a sepsis risk of the present disclosure may further use a risk alarming model configured to determine whether to send an alarm based on biological signal data and/or biological test data for a subject, together with a risk sequence generated by the risk sequence generation model, more specifically a risk score.

As a result, the device for predicting a mortality risk or a sepsis risk of the present disclosure is capable of reducing false alarms when compared to provision of alarms simply based on the critical level of the risk sequence. That is, the device for predicting a mortality risk or a sepsis risk of the present disclosure using the risk alarming model is capable of providing an alarm at the time when an action is required for a subject by further considering various data.

FIG. 5C illustrates prediction results concerning mortality, based on the device for predicting a mortality risk or a sepsis risk according to an exemplary embodiment of the present disclosure. FIG. 5D illustrates prediction results concerning the onset of sepsis, based on the device for predicting a mortality risk or a sepsis risk according to an exemplary embodiment of the present disclosure.

Referring to (a) and (b) of FIG. 5C, area under the curve (AUC) levels and AP (Average Precision) value concerning prediction of mortality performed by the risk sequence generation model of the present disclosure are shown. More specifically, it is shown in (a) of FIG. 5C that as a result of assessment based on the data obtained from Severance ICU, the AUC value is 0.992 and the AP value is 0.909 at a high level according to the prediction of mortality performed 12 hours before mortality by the risk sequence generation model of the present disclosure. At this point, the AUC value may refer to an accuracy rate, which is associated with excellent diagnosability. Referring to (b) of FIG. 5C, it is shown that as a result of assessment based on the data obtained from MIMIC-III, the AUC value is 0.849 and the AP value is 0.515 according to the prediction of mortality performed 12 hours before mortality by the risk sequence generation model used in the device for predicting a risk of the present disclosure.

That is, the risk sequence generation model used in various exemplary embodiments of the present disclosure may be used for not only generating a risk sequence but also predicting a mortality risk.

Referring to (a) and (b) of FIG. 5D, AUC levels and AP value concerning prediction of sepsis onset performed by the risk sequence generation model of the present disclosure are shown. More specifically, it is shown in (a) of FIG. 5D that as a result of assessment based on the data obtained from Severance ICU, the AUC value is 0.865 and the AP value is 0.276 according to the prediction of sepsis onset performed 6 hours before the onset of sepsis by the risk sequence generation model of the present disclosure. Referring to (b) of FIG. 5D, it is shown that as a result of assessment based on the data obtained from MIMIC-III (more specifically evaluating the model that has learned from Severance ICU based on the MIMIC-III data), the AUC value is 0.679 and the AP value is 0.166 according to the prediction of sepsis onset performed 6 hours before sepsis onset by the risk sequence generation model used in the device for predicting a risk of the present disclosure.

That is, the risk sequence generation model used in various exemplary embodiments of the present disclosure may be used for predicting not only a mortality risk but also a sepsis onset risk.

### EXAMPLE 2

### Second Evaluation of Risk Sequence Generation Model Applied to Various Exemplary Embodiments of Present Disclosure

The evaluation was conducted on a risk sequence generation model, which was configured to assess a sepsis risk or a mortality risk based on biological signal data, biological test data, and reference feature data.

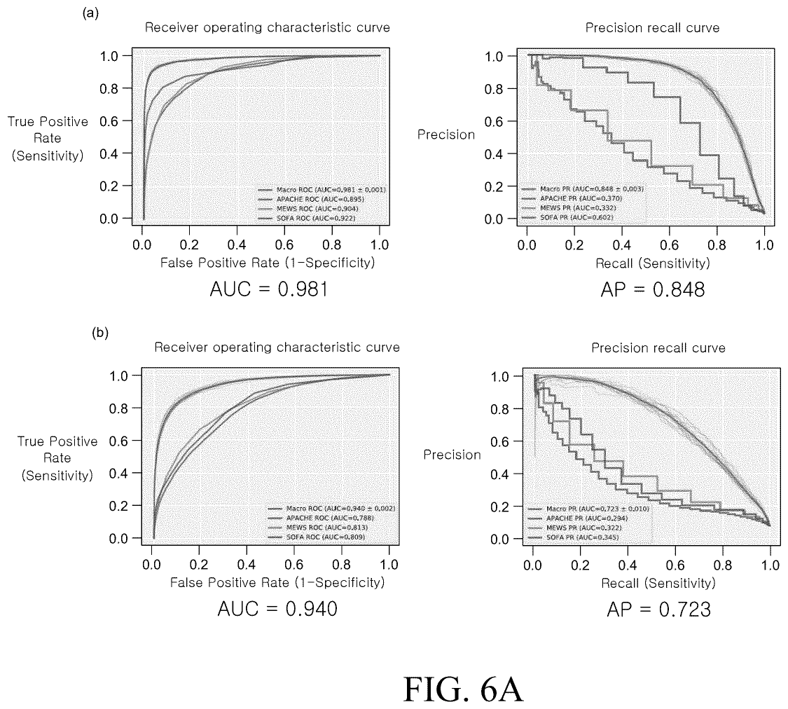

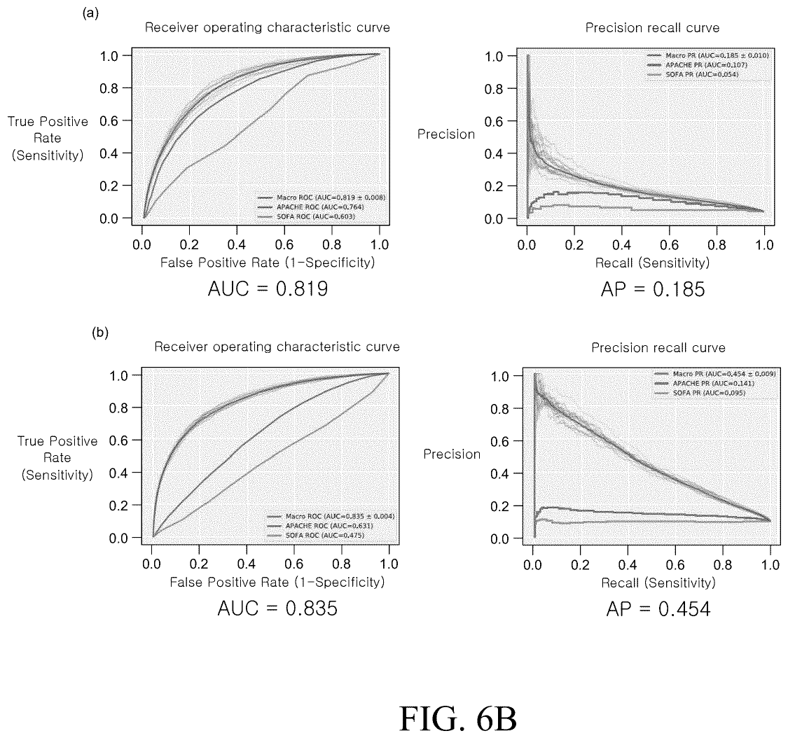

FIG. 6A illustrates prediction results concerning mortality, based on a device for predicting a mortality risk or a sepsis risk according to another exemplary embodiment of the present disclosure. FIG. 6B illustrates prediction results concerning the onset of sepsis, based on the device for predicting a mortality risk or a sepsis risk according to another exemplary embodiment of the present disclosure.

Referring to (a) and (b) of FIG. 6A, AUC levels and precisions concerning prediction of mortality performed by the risk sequence generation model of the present disclosure are shown. More specifically, it is shown in (a) of FIG. 6A that as a result of assessment based on the data obtained from Severance ICU, the AUC value is 0.981 and the AP value is 0.848 at a high level according to the prediction of mortality performed 12 hours before mortality by the risk sequence generation model of the present disclosure. At this point, the AUC value may refer to an accuracy rate, which is associated with excellent diagnosability. Referring to (b) of FIG. 6A, it is shown that as a result of assessment based on the data obtained from MIMIC-III, the AUC value is 0.940 and the AP value is 0.723 according to the prediction of mortality performed 12 hours before mortality by the risk sequence generation model used in the device for predicting a risk of the present disclosure.

That is, the risk sequence generation model used in various exemplary embodiments of the present disclosure may be used for not only generating a risk sequence but also predicting a mortality risk.

Referring to (a) and (b) of FIG. 6B, AUC levels and AP value concerning prediction of sepsis onset performed by the risk sequence generation model of the present disclosure are shown. More specifically, it is shown in (a) of FIG. 6B that as a result of assessment based on the data obtained from Severance ICU, the AUC value is 0.819 and the AP value is 0.185 according to the prediction of sepsis onset performed 6 hours before sepsis onset by the risk sequence generation model of the present disclosure. Referring to (b) of FIG. 6B, it is shown that as a result of assessment based on the data obtained from MIMIC-III (more specifically evaluating the model that has learned from Severance ICU based on the MIMIC-III data, the AUC value is 0.835 and the AP value is 0.454 according to the prediction of sepsis onset performed 6 hours before sepsis onset by the risk sequence generation model used in the device for predicting a risk of the present disclosure.

That is, the risk sequence generation model, which uses as additional learning data the reference feature data including a minimum value, a maximum value, and an average value for each of the biological signal data and the biological test data, may be used to predict not only a mortality risk but also a sepsis onset risk.

Although the exemplary embodiments of the present disclosure have been described in detail with reference to the accompanying drawings, the present disclosure is not limited thereto and may be embodied in many different forms without departing from the technical concept of the present disclosure. Therefore, the exemplary embodiments of the present disclosure are provided for illustrative purposes only but not intended to limit the technical concept of the present disclosure. The scope of the technical concept of the present disclosure is not limited thereto. Therefore, it should be understood that the above-described exemplary embodiments are illustrative in all aspects and do not limit the present disclosure. The protective scope of the present disclosure should be construed based on the following claims, and all the technical concepts in the equivalent scope thereof should be construed as falling within the scope of the present disclosure.

What is claimed is:

1. A method for predicting a mortality risk or a sepsis risk, implemented by a processor, the method comprising:
receiving biological signal data for a subject;
generating a risk sequence for the subject, by using a risk sequence generation model configured to generate a risk sequence based on the biological signal data; and
predicting a mortality risk or a sepsis risk for the subject based on the risk sequence.

2. The method for predicting a mortality risk or a sepsis risk according to claim 1, wherein receiving the biological signal data includes receiving at least one of the biological signal data for the subject selected from the group consisting of a temperature, a pulse, an oxygen saturation, a systolic blood pressure, a diastolic blood pressure, and a mean blood pressure, and
predicting the mortality risk or the sepsis risk includes predicting the mortality risk based on the risk sequence.

3. The method for predicting a mortality risk or a sepsis risk according to claim 2, wherein receiving the biological signal data includes receiving the biological signal data a plurality of times in a predetermined unit of time,
generating the risk sequence includes generating a risk sequence in the predetermined unit of time based on the biological signal data received the plurality of times in the predetermined unit of time, by using the risk sequence generation model, and
predicting the mortality risk or the sepsis risk includes predicting the mortality risk based on the risk sequence in the predetermined unit of time.

4. The method for predicting a mortality risk or a sepsis risk according to claim 1, further comprising receiving biological test data for the subject selected from the group consisting of a Glasgow coma scale (GCS), an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level,
wherein generating the risk sequence further includes generating a sepsis risk sequence based on the biological signal data and the biological test data, by using the risk sequence generation model, and
predicting the mortality risk or the sepsis risk further includes predicting the sepsis risk based on the sepsis risk sequence.

5. The method for predicting a mortality risk or a sepsis risk according to claim 4, wherein receiving the biological test data includes receiving a maximum value, a minimum value, and an average value of the biological test data measured a plurality of times in a predetermined unit of time.

6. The method for predicting a mortality risk or a sepsis risk according to claim 4, wherein receiving the biological signal data includes receiving the biological signal data for the subject selected from the group consisting of a temperature, a pulse, an oxygen saturation, a systolic blood pressure, a diastolic blood pressure, and a mean blood pressure.

7. The method for predicting a mortality risk or a sepsis risk according to claim 1, further comprising, after predicting the mortality risk or the sepsis risk, providing a risk alarm for the subject by using a risk alarming model.

8. The method for predicting a mortality risk or a sepsis risk according to claim 7, further comprising, before providing the risk alarm, receiving biological test data for the subject,
wherein the risk sequence generation model is further configured to calculate a mortality risk score based on the biological signal data, and
wherein the risk alarming model includes:
a first alarming model configured to output a vector value based on the mortality risk score calculated by the risk sequence generation model, a second alarming model configured to output a vector value based on the biological signal data, a third alarming model configured to output a vector value based on the biological test data, or a fourth alarming model configured to determine whether to provide a mortality risk alarm based on the vector value outputted by the first alarming model, the second alarming model, or the third alarming model.

9. The method for predicting a mortality risk or a sepsis risk according to claim 8, further comprising, before providing the risk alarm, receiving drug administration recording or alarm transmission recording for the subject,
wherein the risk alarming model includes the first alarming model and the fourth alarming model,
the first alarming model is further configured to output a vector value based on the mortality risk score, and the drug administration recording or the alarm transmission recording.

10. The method for predicting a mortality risk or a sepsis risk according to claim 1, wherein the mortality risk is defined as a risk of mortality occurrence before a predetermined time, and
the sepsis risk is defined as a risk of sepsis onset before the predetermined time.

11. The method for predicting a mortality risk or a sepsis risk according to claim 1, wherein the risk sequence generation model is a model learning by: receiving learning biological signal data obtained for a specimen subject at a predetermined time before risk occurrence; generating a learning risk sequence based on the learning biological signal data; and predicting a risk for the specimen subject at any time before the risk occurrence based on the learning risk sequence.

12. The method for predicting a mortality risk or a sepsis risk according to claim 11, wherein the specimen subject is a dead subject, and
predicting the risk for the specimen subject includes predicting a mortality risk for the specimen subject based on the learning risk sequence.

13. The method for predicting a mortality risk or a sepsis risk according to claim 11, further comprising receiving learning biological test data obtained for the specimen subject at a predetermined time before sepsis onset,
wherein the specimen subject is a subject suffering from sepsis,
generating the learning risk sequence includes generating a learning sepsis risk sequence based on the learning biological test data and the learning biological signal data, and
predicting the risk for the specimen subject includes predicting a sepsis onset risk for the specimen subject based on the learning sepsis risk sequence.

14. A device for predicting a mortality risk or a sepsis risk, the device comprising:
a receiving unit configured to receive biological signal data for a subject; and
a processor operably connected to the receiving unit for communication,
wherein the processor is configured to
generate a risk sequence for the subject by using a risk sequence generation model configured to generate a risk sequence based on the biological signal data, and predict a mortality risk or a sepsis risk for the subject based on the risk sequence.

15. The device for predicting a mortality risk or a sepsis risk according to claim 14, wherein the receiving unit is further configured to receive biological test data for the subject selected from the group consisting of a Glasgow coma scale, an arterial oxygen saturation, a fraction of inspired oxygen concentration, a bicarbonate ion concentration, a bilirubin level, a creatinine level, a platelet count, a total urine output, a potassium concentration, a sodium concentration, a white blood cell count, a lactate concentration, an anterior pituitary hormone (APH) level, and a hematocrit level, and
wherein the processor is further configured to generate a sepsis risk sequence based on the biological signal data and the biological test data by using the risk sequence generation model, and predict the sepsis risk based on the sepsis risk sequence.

16. The method for predicting a mortality risk or a sepsis risk according to claim 1, wherein receiving the biological signal data includes receiving a maximum value, a minimum value, and an average value of the biological signal data measured a plurality of times in a predetermined unit of time, and
generating the risk sequence for the subject includes generating a risk sequence for the subject based on the maximum value, the minimum value, and the average value of the biological signal data, by using the risk sequence generation model.

17. The method for predicting a mortality risk or a sepsis risk according to claim 1, further comprising receiving age data for the subject,
wherein generating the risk sequence for the subject further includes generating a risk sequence for the subject based on the biological signal data and the age data, by using the risk sequence generation model.

18. The device for predicting a mortality risk or a sepsis risk according to claim 14, wherein the receiving unit is further configured to receive a maximum value, a minimum value, and an average value of the biological signal data measured a plurality of times in a predetermined unit of time, and
the processor is further configured to generate a risk sequence for the subject based on the maximum value, the minimum value, and the average value of the biological signal data, by using the risk sequence generation model.

19. The device for predicting a mortality risk or a sepsis risk according to claim 15, wherein the receiving unit is further configured to receive a maximum value, a minimum value, and an average value of the biological test data measured a plurality of times in a predetermined unit of time, and
the processor is further configured to generate a sepsis risk sequence for the subject based on the maximum value, the minimum value, and the average value of the biological test data, by using the risk sequence generation model.

20. The device for predicting a mortality risk or a sepsis risk according to claim 14, wherein the receiving unit is further configured to receive age data for the subject, and
The processor is further configured to generate a risk sequence for the subject based on the biological signal data and the age data, by using the risk sequence generation model.
